(19) United States
(12) Patent Application Publication
(10) Pub. No.: US 2011/0064669 A1
(43) Pub. Date: Mar. 17, 2011

(54) USE OF GALANIN IN A METHOD OF TREATING NEURODEGENERATIVE DISEASES OR CONDITIONS

(75) Inventors: TREVOR KILPATRICK, PARKVILLE VIC (AU); HELMUT BUTZKUEVEN, KENSINGTON VIC (AU); VICTORIA PERREAU, COBURG VIC (AU); PIK YING SOO, MELAKA (MY)

(73) Assignee: HOWARD FLOREY INSTITUTE OF EXPERIMENTAL PHYSIOLOGY AND MEDICINE, PARKVILLE VIC (AU)

(21) Appl. No.: 12/829,812

(22) Filed: Jul. 2, 2010

Related U.S. Application Data
(63) Continuation of application No. 12/523,663, now abandoned.

(30) Foreign Application Priority Data
Jan. 19, 2007 (AU) 2007900278

(51) Int. Cl. A61K 38/22; A61K 38/17; A61B 5/055; A61P 25/00

(52) U.S. Cl. 424/9.2; 514/17.7; 514/17.9; 514/9.7

(57) ABSTRACT

The present invention relates generally to a method for the treatment and prophylaxis of neurodegenerative diseases and conditions. More particularly, the present invention contemplates the treatment or prophylaxis of neurological conditions involving oligodendrocyte cytotoxicity or cell cycle arrest, demyelination and/or axonal or neuronal degeneration. Agents, medicaments and pharmaceutical compositions useful in the treatment and prophylaxis of neurodegenerative conditions and disorders also form part of the present invention.

### RELATED APPLICATIONS

This application is a Continuation of U.S. application Ser. No. 12/523,663, filed Jul. 17, 2009, which is the U.S. National Phase of International Application No.: PCT/AU2008/000054, filed Jan. 16, 2008, designating the U.S. and published in English on Jul. 24, 2008 as WO 2008/086573, which claims the benefit of Australian Application No. 2007900278, filed Jan. 19, 2007.

### FIELD

The present invention relates generally to a method for the treatment and prophylaxis of neurodegenerative diseases and conditions. More particularly, the present invention contemplates the treatment or prophylaxis of neurological conditions involving oligodendrocyte cytotoxicity or cell cycle arrest, demyelination and/or axonal or neuronal degeneration. Agents, medicaments and pharmaceutical compositions useful in the treatment and prophylaxis of neurodegenerative conditions and disorders also form part of the present invention.

### BACKGROUND

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in any country.

Neurodegenerative diseases are particularly debilitating conditions resulting from the destruction of components of the nervous system. Multiple sclerosis (MS), for example, is a particularly destructive inflammatory disease process resulting in the immune-based destruction of myelin surrounding the nerves of the central nervous system (CNS).

Current treatment for MS includes β-interferon and glatiramer acetate which act by modulating immune activity. However, whilst these therapies have some capacity to reduce disease activity, they have limited capacity to reduce long-term disability. There is a need to establish treatments that act directly either to reduce nervous system damage or to enhance its repair.

Given that MS pathology is principally the temporally and spatially distinct loss of myelin and oligodendrocytes, oligodendrocytes themselves represent a therapeutic target. Interestingly, the fate of the oligodendrocytes within demyelinating lesions is not uniform. Mature oligodendrocytes in different plaques may die in large numbers by apoptosis, or alternatively, survive as demyelinated, mature cells (Lucchinetti et al, Brain 122:2279-2295, 1999; Lucchinetti et al, Ann Neurol 47:707-717, 2000). Subsequently, re-myelination may be complete, partial or entirely absent, as first described by Suzuki et al, Lab Invest 20:444-454, 1969.

The endogenous molecular processes responsible for both mature oligodendrocyte survival and re-myelination in vivo are poorly described. However, the discovery of several cytokines with the capacity to promote survival of purified rat oligodendroglia has revealed some potentially relevant candidates including insulin-like growth factor-1 (IGF-1) [Barres et al, Cell 70:31-46, 1992; Barres et al, Development 118:283-295, 1993], glial growth factor 2 (GGF-2) [Canon et al, Neuron 17:229-243, 1996; Fernandez et al, Neruon 28:81-90, 2000], neurotrophin-3 (NT-3) [Barres et al, supra 1993], and interleukin-6 (IL-6) family members such as ciliary neurotrophic factor (CNTF) [Mayer et al, Development 120:143-153, 1994]. These cytokines may inhibit oligodendrocyte cell death induced by tumor necrosis factor-α (TNF-α) [D'Souza et al, J Neurosci Res 43:289-298, 1996] or γ-interferon (IFN-γ) [Vartanian et al, Proc Natl Acad Sci USA 96:731-735, 1999].

Experimental autoimmune encephalomyelitis (EAE) is a disease induced by immunization against various myelin epitopes, producing multifocal inflammation, demyelination and oligodendrocyte loss in the CNS. Administration of recombinant leukemia inhibitory factor (LIF) reduces EAE severity by promoting oligodendrocyte survival and reducing myelin destruction, without any demonstrable concomitant anti-inflammatory activity (Butzkeuven et al, Nat Med 8:613-619, 2002). Interestingly, oligodendrocytes near inflammatory infiltrates up-regulate LIF receptor beta (LIFRβ) [Butzkeuven et al, supra 2002] and, upon LIF administration, exhibit enhanced STAT3 phosphorylation, suggesting that the site of action of recombinant LIF is the inured oligodendrocyte, itself.

It has also been demonstrated that compound heterozygous mutant mice, carrying a deletion of LIFRβ (Ware et al, Development 121:1283-1299, 1995), as well as a deletion of the four STAT3 binding phosphor-tyrosines of gp130 (Ernst et al, J Exp Med 194:189-203, 2001), exhibited a marked reduction in mature oligodendrocyte survival in the context of EAE, accompanied by more severe demyelination and a fatal disease course (Butzkeuven et al, supra 2002). A very similar phenotype was described in CNTF knockout mice challenged with EAE (Linker et al, Nat Med 8:620-624, 2002). It was demonstrated that LIF is produced in the spinal cord of mice suffering acute EAE. Blocking this cytokine response with anti-LIF neutralizing antibodies worsens clinical disease, increasing both demyelination and oligodendrocyte loss, but does not affect the extent of inflammatory infiltration into the CNS (Butzkeuven et al, Glia 53:696-703, 2006). Endogenous LIF signaling also limits non-immune mediated demyelination. LIF knockout mice exhibit more severe demyelination when challenged with cuprizone, a well-established model of toxin-mediated demyelination (Emery et al, Proc Natl Acad Sci USA 103 (20): 7859-7864, 2006). Exogenous LIF can also reduce the severity of demyelination in wild-type litter mates (Marriott et al, LIE receptor signaling modulates myelin loss and repair during cuprizone demyelination, Poster Abstract, The Australian Neuroscience Society 26^{th} Annual meeting, 2006).

An emergent view is that MS is actually an example of an inflammatory neurodegenerative disease in which the nervous system damage is characterized by both demyelination and axonal degeneration (Trapp et al, N Engl J Med 338:278-285, 1998). In addition, demyelinating lesions can be identified within the cortex and neuronal loss has also been shown to occur within these regions (Vercellino et al, J Neropathol Exp Neurol. 64:1101-1107, 2005). It is, therefore, of potential relevance that it has also been found that endogenous LIF receptor signaling is also important for neuronal survival/maintenance which could potentially prove relevant to limiting the neuronal pathology in MS. After a surgical cortical lesion in adult rat brain, LIF mRNA expression, quantitated using RNAse protection, increases thirty-fold within six hours of the injury, and reaches a peak at 24 hours (Banner et al, Exp Neurol 147:1-9, 1997). The major cells responsible for the production of LIF mRNA in this model are GFAP positive astrocytes. Axotomy (trans-section of the nerve cell axon) represents a significant neuronal injury. In adult animals, this insult may be sublethal in some neuronal populations, but lethal in other experimental models.

Collectively, these data demonstrate that endogenous LIF receptor signaling directly promotes oligodendrocyte survival in the context of demyelinating disease. However, LIF is a highly pleiotropic molecule and, hence, it is not necessarily an ideal candidate on its own for use in neurodegenerative diseases.

There is a need, therefore, to develop alternative therapies which prevent or reduce oligodendrocyte cytotoxicity and/or cell cycle arrest and associated demyelination and axonal and neural degeneration.

### SUMMARY

The present invention provides a method for the treatment or prophylaxis of neurodegenerative disease or conditions, in particular inflammatory neurodegenerative disease or conditions and more particularly neurodegenerative conditions involving oligodendrocyte cytotoxicity or cell cycle arrest, demyelination and/or axonal or neuronal degeneration. In accordance with the present invention, elevating levels of galanin or homologs, derivatives, analogs or mimetics thereof or of a galanin receptor is proposed to potentiate oligodendrocyte survival and to inhibit demyelination and/or promote axonal and neuronal repair and function. This in turn is useful in ameliorating or reducing the effects of inflammatory neurodegeneration such as in MS, acute disseminated encephalomyelitis, optic neuropathy (including neuromyelitis optic with transient autonomic disturbances), Devic's neuromyelitis optica, tropical spastic paraparesis, non-compressive myelopathies, concentric sclerosis, diffuse sclerosis acute hemorrhagic leukoencephalopathy, metabolic leukodystrophy leucoarliusis, acute discriminated encephalomyelitis, progressive multifocal leukoencephalopathy, multisystem entrophy and repairing the demyelination associated with disease or trauma.

Accordingly, the present invention contemplates a method for the treatment or prophylaxis of a neurodegenerative disease or condition in a subject, said method comprising administering to said subject an effective amount of galanin or homolog, derivative, analog or mimetic thereof and/or an agent which up-regulates the level or activity of a galanin receptor for a time and under conditions sufficient to promote survival of oligodendrocytes, inhibit demyelination and/or promote axonal and/or neuronal repair and function.

Galanin or its homolog, derivative, analog or mimetic or the agent which up-regulates the level or activity of a galanin receptor (i.e. a galanin receptor agonist) may be provided alone or in combination with another neuroprotective agent including an oligodendrocyte survival-, myelination- and/or axonal and/or neuronal-promoting agent such as but not limited to LIF and/or CNTF or a functional equivalent, homolog, derivative, analog or mimetic.

Hence, the present invention contemplates combination therapy for neurodegenerative disorders involving oligodendrocyte cytotoxicity or cell cycle arrest and/or demyelination and/or axonal or neuronal degeneration said combination therapy comprising the administration of galanin or a homolog, derivative, analog or mimetic thereof and/or a galanin receptor agonist and one or both of LIF and/or CNTF or a functional equivalent, homolog, derivative, analog or mimetic thereof.

Compositions comprising two or more of galanin, a galanin receptor agonist, LIF and/or CNTF or their homologs, derivatives, analogs or mimetics also form part of the present invention.

The present invention further provides for the use of galanin or its homolog, derivative, analog or mimetic or a galanin receptor agonist alone or in combination with LIF or CNTF in the manufacture of a medicament for the treatment of a neurodegenerative disease in a subject.

The preferred subject is a human subject.

A diagnostic or screening test involving magnetic resonance imaging (MRI) on the optic nerve is also part of the present invention.

A list of abbreviations used throughout the subject specification is provided in Table 1.

TABLE 1
Abbreviations

| ABBREVIATION | DESCRIPTION |
|---|---|
| CC1 | Oligodendrocyte specific antibody |
| CNS | Central nervous system |
| CNTF | Ciliary neurotrophic factor |
| EAE | Experimental autoimmune encephalomyelitis |
| GGF-2 | Glial growth factor 2 |
| IFN-β | Beta-interferon |
| IFN-γ | Gamma-interferon |
| IGF-1 | Insulin-like growth factor |
| IL-6 | Interleukin-6 |
| LIF | Leukemia inhibitory factor |
| LIFR | Leukemia inhibitory factor receptor |
| LIFRβ | Beta component of LIFR |
| MRI | Magnetic resonance imaging |
| NT-3 | Neurotrophin-3 |
| OC | Oligodendrocyte |
| PDGF | Platelet-derived growth factor |
| SG-2 | Secretogranin-II |
| TNF-α | Tumor necrosis factor-alpha |
| WT | Wild-type |

### BRIEF DESCRIPTION OF THE FIGURES

Some figures contain color representations or entities. Color photographs are available from the Patentee upon request or from an appropriate Patent Office. A fee may be imposed if obtained from a Patent Office.

FIG. 1 is a photographic representation showing a sagittal localizer for imaging optic nerves (left panel, the line indicates the position of optic nerves) followed by oblique acquisition of single slice imaging optic nerve (right panel). White arrow indicates the left optic nerve.

FIG. 2 is a photographic representation showing the diffusion weighted image (high diffusion=low signal) applied parallel (left panel) and perpendicular (right panel) to the left optic nerve. The scans indicate that the apparent diffusion coefficient is greater in parallel (left panel, lower signal) than perpendicular to the nerve (right panel, higher signal).

FIG. 3 is a graphical representation showing parallel optic nerve ADC (Para) in EAE mice (mean 1250, sd 163, n=20) is significantly lower than in healthy mice (mean 1586, sd 139, n=10), p<0.0001. The Perpendicular ADC value (Perp) is around 50% of parallel ADC, and not changed significantly in EAE compared with healthy mice.

FIG. 4 is a photographic representation showing the longitudinal frozen sections of optic nerves in EAE revealing evidence of optic neuritis. Top panels show inflammatory infiltrates evident in DAPI nuclear stained sections (white arrows). Lower panels illustrate axonal injury, revealed by immuno-staining for hypo-phosphorylated neurofilament (white arrows). The lower panels also suggest an EAE associated reduction in optic nerve size, subsequently confirmed by cross-sectional area measures.

FIG. 5 is a photographic representation showing cross-sections of optic nerve examined by electron microscope. A healthy nerve is shown in the left panel and a nerve from an animal suffering EAE in the right panel. In spite of overt cellular infiltration, only one demyelinated axon is visible.

FIG. 6 is a photographic representation showing Panel A: typical low power image of optic nerve in cross-section (20 times magnification). The central box indicates the size of Panels B-D (100 times magnification). Panel B: methylene-blue stained optic nerve central cross-section, Panel C: image imported as grey-scale into Image J with cells manually masked. Panel D: thresh-holding axolemma.

FIG. 7 is a graphical representation showing axolemmal cross-sectional area size distribution revealing EAE-associated axonal atrophy. The change in the percentage of large axons (>19 square micrometers) is highly significant (p=0.01). The graph represents the average of axonal size frequencies in 5 healthy optic nerves and 10 EAE optic nerves.

FIG. 8 is a graphical representation showing the MRI measurement of parallel ADC and the total axolemmal area in optic nerve cross-sections which are highly correlated, r=0.86, p<0.01. X-axis units in square mm. Red diamonds represent the healthy controls, blue diamonds animals with severe EAE.

FIG. 9 is a graphical representation showing normalised representation of identified gene clusters regulated by LIF (left panel). The right panel shows the identify of several LIF-regulated genes (8 hours of LIF exposure) including a major structural component of secretory granules (secretogranin II; SG-2) and various cytokines/neuropeptides. Results represent the average regulation in the LIF condition versus the NT3, Insulin or Sato only conditions (nine comparisons in total), error bars represent SD. All are significantly regulated, p<0.001. X-axis=fold-change.

FIG. 10 is a graphical representation showing confirmation of induction of SG-2 and galanin mRNA in OPCs and four independent OC cultures, exposed to LIF (100 ng/ml) for 5 (Oligo 1/2) and 24 hours (Oligo 3/4). The graphs illustrate expression relative to independent baseline conditions (baseline 1v1=1), without LIF.

FIG. 11 is a photographic representation showing LIF exposure stimulating galanin secretion in concentrated media from oligodendrocytes (OC) [top panel] and CG4 cells (bottom panel). The detected band is 16 kD in size, equivalent to the reported size of secreted preprogalanin. The protein content in each lane was 10 microgram, measured using a Pierce BCA kit.

FIG. 12 is a photographic representation showing LIF-treated OC (right panels) displaying intense polar galanin staining in comparison to untreated OC (left panels). The staining suggests a granular cytoplasmic localization of galanin protein, most likely in the Golgi apparatus. The red staining identifies CNPase positivity, an oligodendroglial marker and the cell nuclei are stained blue with DAPI.

FIG. 13 is a graphical representation showing the survival of post-mitotic OC in vitro increased in the presence of a low concentration of galanin (left panel). The difference between 1 nM galanin and all other conditions is highly significant, p<0.01. No other galanin concentration is significantly different from baseline (nil). The right panel illustrates lack of synergy between LIF and galanin. There is no difference in OC survival in the presence of galanin alone or galanin plus LIF. OC survival in the presence of galanin and galanin plus LIF is significantly different to Sato alone (nil) or Sato plus LIF, p<0.01 for each comparison.

FIG. 14 is a photographic representation showing galanin staining in PLPdsRED positive OC induced by cuprizone challenge. Left panel: galanin-staining of neurons and neuronal processes in periaqueductal grey matter, no staining of PLPdsRED positive cells. Middle panel: galanin is induced in PLPdsRED positive OC (white asterisks) in the corpus callosum after three weeks of cuprizone challenge. Almost all galanin-positive cells are PLPdsRED positive. The right panel illustrates the red channel image of the same corpus callosal region.

FIG. 15 is a photographic representation showing galanin-positive oligodendrocytes specific antibody (CCI) induced by cuprizone challenge in wild-type (right panel) but not LIF knockout (left panel) corpus callosum.

FIG. 16 is a graphical representation showing 78% of CC1 positive oligodendrocytes in wild-type (WT) corpus callosum expressed galanin during cuprizone challenge. Galanin positivity in OC is markedly reduced in cuprizone-challenged LIF ko (18%, p=0.08 vs WT) and LIF/CNTF double mice (14%, p=0.04 vs WT). CNTF knockout mice are not significantly different from wild-type mice (p=0.47).

FIG. 17 is a photographic representation showing EAE in PLPdsRED transgenic mouse, lateral spinal cord (20 times magnification), left panel. Red oligodendrocytes are visualized, some within/surrounding a large inflammatory infiltrate (arrowheads). Middle panel: PLPdsRED positive cells near infiltrate express galanin granules (arrowheads) [100 times magnification]. Right panel: dsRED positive cell within inflammatory infiltrate (DAPI blue nuclear stain) expresses galanin, 100 times magnification.

### DETAILED DESCRIPTION

All scientific citations, patents, patent applications and manufacturer's technical specifications referred to hereinafter are incorporated herein by reference in their entirety.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It is to be understood that unless otherwise indicated, the subject invention is not limited to specific formulation components, manufacturing methods, dosage regimens, or the like, as such may vary. It is also to be understood that the terminology used herein is for the purpose of describing particular embodiments only and is not intended to be limiting.

It must be noted that, as used in the subject specification, the singular forms “a”, “an” and “the” include plural aspects unless the context clearly dictates otherwise. Thus, for example, reference to “a target” includes a single target, as well as two or more targets; reference to “an oligodendrocyte” includes a single oligodendrocyte, as well as two or more oligodendrocytes; reference to “the formulation” includes a single formulation, as well as two or more formulations; and so forth.

In describing and claiming the present invention, the following terminology is used in accordance with the definitions set forth below.

The terms “compound”, “active agent”, “chemical agent”, “pharmacologically active agent”, “medicament”, “active” and “drug” are used interchangeably herein to refer to a chemical compound including a protein or nucleic acid molecule that induces a desired pharmacological and/or physiological effect. The desired effect includes inducing or promoting oligodendrocyte survival and maintenance, promotion or protection of myelination and/or protection of axons and/or neurons. The desired physiological effect also includes an observation of amelioration of symptoms of neurodegenerative disease such as amelioration of symptoms of MS or of EAE in an animal model. The terms also encompass pharmaceutically acceptable and pharmacologically active ingredients of those active agents specifically mentioned herein including but not limited to salts, esters, amides, prodrugs, active metabolites, analogs, mimetics functional equivalents and the like. When the terms “compound”, “active agent”, “chemical agent” “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc. Hence, reference to agents such as “galanin”, “LIF” and “CNTF” includes their homologs, derivatives, analogs and mimetics as well as pharmaceutically acceptable, pharmacologically active salts, esters, amides, prodrugs, metabolites, etc.

Reference to a “compound”, “active agent”, “chemical agent” “pharmacologically active agent”, “medicament”, “active” and “drug” includes combinations of two or more actives such as galanin and/or galanin receptor agonist and LIF and/or CNTF. A “combination” also includes multi-part such as a two-part composition where the agents are provided separately and given or dispensed separately or admixed together prior to dispensation. For example, a multi-part pharmaceutical pack may have two or more of galanin and LIF or CNTF separately maintained.

Reference to promoting oligodendrocyte survival includes reducing oligodendrocyte cytotoxicity or cell cycle arrest as well as promoting oligodendrocyte maintenance; promoting or protecting myelination includes inhibiting, preventing or otherwise reducing demyelination; protection of axons and neurons includes promoting axonal and neuronal repair, function and maintenance. The term “cell cycle arrest” includes cytostasis or other arrest of cell growth (whether cytotoxic or not) and cell senescence.

Reference to particular cytokines or growth factors or neuroprotective peptides such as LIF, CNTF and galanin include their homologs, derivatives, chemically modified forms or analogs, mimetics and functional equivalents. For example, galanin is a 29 amino acid peptide. In order to enhance its serum or cerebral half-life or its penetrability of the blood brain barrier, any one or more of the 29 amino acid residues may be substituted by a non-naturally occurring amino acid analog such as those listed in Table 2.

Alternatively or in addition, amide bonds between amino acid residues may be chemically modified or side chains added or modified. This applies not only to galanin but also cytokines such as LIF and CNTF.

Analogs of the agents contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecule or their analogs. This term also does not exclude modification of the glycosylation, acetylation and phosphorylation patterns. Included within the definition are, for example, polypeptides containing one or more analogs of an amino acid (including, for example, unnatural amino acids such as those given in Table 2) or polypeptides with substituted linkages. Such polypeptides may need to be able to enter the cell and/or cross the blood-brain barrier or promote serum or cerebral or tissue fluid half-life survival.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids, contemplated herein is shown in Table 2.

TABLE 2
Codes for non-conventional amino acids

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl)carbamylmethyl)glycine | Nnbhm | N-(N-(3,3-diphenylpropyl)carbamylmethyl)glycine | Nnbhe |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

Mimetics of the subject agents (i.e. galanin, LIF and CNTF) are another useful group of agents to test for neuroprotective ability. The term is intended to refer to a substance which has some chemical similarity to the molecule it mimics and which acts as an agonist. A peptide mimetic of galanin, for example, may be a peptide-containing molecule that mimics elements of protein secondary structure (Johnson et al, Peptide Turn Mimetics in Biotechnology and Pharmacy, Pezzuto et al (Eds), Chapman and Hall, New York, 1993). The underlying rationale behind the use of peptide mimetics is that the peptide backbone of proteins exists chiefly to orient amino acid side chains in such a way as to facilitate molecular interactions such as with a receptor or ligand. A peptide mimetic, therefore, is designed to permit molecular interactions similar to the natural molecule.

The designing of mimetics to a pharmaceutically active compound is a known approach to the development of pharmaceuticals based on a “lead” compound. This might be desirable where the active compound is difficult or expensive to synthesize or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g. by substituting each residue in turn. Alanine scans of peptides, for example, are commonly used to refine such peptide motifs. These parts or residues constituting the active region of the compound are known as its “pharmacophore”.

Once the pharmacophore has been found, its structure is modeled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modeling process.

In a variant of this approach, the three-dimensional structure of a receptor and ligand are modeled. This can be especially useful where the receptor and/or ligand change conformation on binding, allowing the model to take account of this in the design of the mimetic. Modeling can be used to generate agents which interact with the linear sequence or a three-dimensional configuration.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

The goal of rational drug design is to produce structural analogs of biologically active polypeptides of interest or of small molecules with which they interact (e.g. agonists, antagonists, inhibitors or enhancers) in order to fashion drugs which are, for example, more active or stable forms of the polypeptide, or which, for example, enhance or interfere with the function of a polypeptide in vivo (see, e.g. Hodgson, Bio Technology 9:19-21, 1991). In one approach, one first determines the three-dimensional structure of a protein of interest by x-ray crystallography, by computer modeling or most typically, by a combination of approaches. Useful information regarding the structure of a polypeptide may also be gained by modeling based on the structure of homologous proteins.

The terms “effective amount” and “therapeutically effective amount” of an agent as used herein mean a sufficient amount of an agent (e.g. galanin or LIF or CNTF or galanin receptor agonist) to provide the desired therapeutic or physiological effect or outcome as indicated above. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, emide, prodrug or derivative of a compound as provided herein is a salt, ester, amide, prodrug or derivative that this not biologically or otherwise undesirable.

The terms “treating” and “treatment” as used herein refer to reduction in severity and/or frequency of symptoms of the condition being treated, elimination of symptoms and/or underlying cause, prevention of the occurrence of symptoms of the condition and/or their underlying cause and improvement or remediation or amelioration of damage following a neurodegenerative condition. In general terms, treatment may involve actively reversing a disease or ameliorating symptoms of, for example, oligodendrocyte cell death, senescence or arrest of cell growth, demyelination and/or axonal or neuronal degeneration. Amelioration of downstream physiological, psychological or mental conditions is also a useful indicator of treatment.

“Treating” a subject, therefore, may involve prevention of a condition or other adverse physiological or psychological event in a susceptible individual as well as treatment of a clinically symptomatic individual by ameliorating the symptoms of the condition.

A “subject” as used herein refers to an animal, preferably a mammal and more preferably human who can benefit from the pharmaceutical agents and formulations and methods of the present invention. There is no limitation on the type of animal that could benefit from the presently described pharmaceutical formulations and methods. A subject regardless of whether a human or non-human animal may be referred to as an individual, patient, animal, host or recipient. The compounds and methods of the present invention have particular applications in human medicine.

As indicated above, the preferred animals are humans but other primates such as orangutangs, gorillas and marmosets, macaques, livestock animals, laboratory test animals, companion animals or captive wild animals, as well as avian species may be useful animal models.

Examples of laboratory test animals include mice, rats, rabbits, guinea pigs and hamsters. Rabbits and rodent animals, such as rats and mice, provide a convenient test system or animal model. Livestock animals include sheep, cows, pigs, goats, horses and donkeys. Non-mammalian animals such as avian species, zebrafish, and amphibians including Xenopus spp.

Whilst humans are the most important subject, non-human animals are useful animal models. In one embodiment, EAE may be induced in a non-human animal as a model to test potential neuroprotective agents.

The term “oligodendrocyte” or its plural form “oligodendrocytes” means those neural cells which provide support for axons and which produce the myelin sheath. Oligodendrocytes form segments of myelin sheaths of numerous neurons. Oligodendrocytes are a class of glial cells. The effects of the subject agents may manifest on the oligodendrocytes themselves as well as on related cells (e.g. other glial cells), precursor cells or progeny or more mature cells.

In accordance with the present invention, it is proposed to employ neuroprotective agents to reduce oligodendrocyte cytotoxicity or cell cycle arrest or to promote oligodendrocyte maintenance due to degenerative inflammatory processes such as immunodegenerative processes or conditions which induce cell cycle arrest.

In addition, the neuroprotective agent may reduce demyelination or promote or maintain myelination processes and/or prevent axonal or neuronal degeneration or promote axonal or neuronal repair. One neuroprotective agent of the present invention is proposed to be galanin or its homologs, derivatives, analogs or mimetics.

Another neuroprotective agent is proposed to be an agent which up-regulates the levels or activity of a galanin receptor. Such an agent is referred to herein as a galanin receptor agonist. Reference to a “galanin receptor” includes galanin receptor I and galanin receptor II. The present invention further extends to combinations of neuroprotective agents or neuroprotective formulations comprising one or both of galanin or its homologs, derivatives, analogs or mimetics and/or a galanin receptor agonist and another neuroprotective agent such as LIF or CNTF or a homolog, derivative, analog or mimetic thereof. In addition, the present invention extends to other components in the LIF/CNTF signaling pathway. Reference to all molecules (e.g. galanin, LIF, CNTF), receptors (e.g. galanin receptor 1 or 2) or genes encoding same include any polymorphic or splice variants thereof. Galanin receptor 2 is particularly preferred.

Hence, the present invention contemplates a method for the treatment or prophylaxis of a neurodegenerative disease or condition in a subject, the method comprising administering to the subject an effective amount of galanin or homolog, derivative, analog or mimetic thereof and/or an agent which up-regulates the level or activity of a galanin receptor for a time and under conditions sufficient to promote survival of oligodendrocytes, inhibit demyelination and/or promote axonal and neuronal repair and function.

Another aspect of the present invention provides a method for the treatment of a neurodegenerative disease in a subject or at least delaying onset of symptoms thereof, the method comprising administering to the subject an effective amount of galanin or an agent which up-regulates the level or activity of a galanin receptor for a time and under conditions sufficient to promote survival of oligodendrocytes, inhibit demyelination or promote axonal and neuronal repair and function.

The present invention further contemplates the treatment or prophylaxis of a neurodegenerative disease or condition resulting from one or more of oligodendrocyte cytotoxicity or cell cycle arrest, demyelination and/or disruption to axons or neurons in a subject, the method comprising administering to the subject an effective amount of a neuroprotective agent comprising galanin or a homolog, derivative, analog or mimetic thereof and/or a galanin receptor agonist for a time or under conditions sufficient to promote oligodendrocyte survival and/or maintenance.

As indicated above, the neuroprotective agent may be a neuroprotective formulation comprising one or both of galanin and/or a galanin receptor agonist and one or both of LIF and/or CNTF.

Hence, the present invention provides a method for the treatment or prophylaxis of a neurodegenerative disease or condition in a subject, the method comprising administering to the subject an effective amount of a neuroprotective formulation comprising one or both of galanin or a homolog, derivative, analog or mimetic thereof and/or a galanin receptor agonist and one or both of LIF and CNTF or a homolog, derivative, analog or mimetic thereof for a time and under conditions sufficient to promote survival and/or maintenance of oligodendrocytes, inhibit demyelination and/or promote axonal and neuronal repair and function.

Still another aspect of the present invention is directed to a method for the treatment of a neurodegenerative disease in a subject or delaying onset of symptoms thereof, the method comprising administering to the subject an effective amount of a neuroprotective formulation comprising galanin or a galanin receptor agonist and one or both of LIF and CNTF for a time and under conditions sufficient to promote survival of oligodendrocytes, inhibit demyelination or promote axonal and neuronal repair and function.

As indicated above, the amount or time sufficient to treat the neurodegenerative disease or condition may be the amount or time required to ameliorate one or more symptoms of the neurodegenerative disorder. A symptom includes a psychological or mental symptom.

Neurodegenerative diseases and conditions contemplated herein include MS, acute disseminated encephalomyelitis, optic neuropathy (including neuromyelitis optic with transient autonomic disturbances) Devic's neuromyelitis optica, tropical spastic paraparesis, non-compressive myelopathies, concentric sclerosis, diffuse sclerosis acute hemorrhagic leukoencephalopathy, metabolic leukodystrophy leucoarliusis, acute discriminated encephalomyelitis, progressive multifocal leukoencephalopathy, multisystem entrophy and repairing the demyelination associated with disease or trauma.

MS is a particularly important condition and is a preferred embodiment of the present invention.

Hence, the present invention contemplates a method for treating MS in a subject, said method comprising administering to said subject an effective amount of galanin or a homolog, derivative, analog or mimetic thereof and/or a galanin receptor agonist for a time and under conditions sufficient to ameliorate the symptoms of MS and/or to promote survival and/or maintenance of oligodendrocytes, inhibit demyelination and/or promote axonal or neuronal repair or function.

The galanin and/or galanin receptor agonist may also be provided in combination with another neuroprotective agent such as LIF and/or CNTF or their homologs, derivatives, analogs or mimetics.

Hence, the present invention provides a method for the treatment or prophylaxis of MS in a subject, said method comprising administering to said subject an effective amount of a neuroprotective formulation comprising one or both of galanin or a homolog, derivative, analog or mimetic thereof and/or a galanin receptor agonist and one or both of LIF and CNTF or a homolog, derivative, analog or mimetic thereof for a time and under conditions sufficient to promote survival and/or maintenance of oligodendrocytes, inhibit demyelination and/or promote axonal and neuronal repair and function.

Reference to “MS in a subject” includes treating a subject with MS, potentially with MS or at risk of developing MS.

Another aspect of the present invention contemplates a method for the treatment of a MS in a subject, the method comprising administering to the subject an effective amount of a neuroprotective formulation comprising galanin or a galanin receptor agonist and one or both of LIF and CNTF for a time and under conditions sufficient to promote survival of oligodendrocytes, inhibit demyelination or promote axonal and neuronal repair and function.

Whilst chemical or proteinaceous agents are particularly preferred in accordance with the present invention, the present invention may also be practiced using a genetic approach.

Accordingly, the present invention contemplates a method for the treatment or prophylaxis of a neurodegenerative disease or condition in a subject, said method comprising administering to said subject an effective amount of a genetic agent which increases levels of galanin and/or a galanin receptor, alone or in combination with LIF and/or CNTF.

The genetic agents include, for example, viral constructs which introduce cDNA or mRNA which encode galanin, a galanin receptor, LIF or CNTF; naked cDNA or mRNA encoding galanin, a galanin receptor, LIF or CNTF; and a RNAi or antisense construct which down-regulate inhibitors of genes encoding galanin, a galanin receptor, LIF or CNTF.

As indicated above, the preferred subject is a human and an example of a neurodegenerative disease or condition is MS.

Hence, the present invention also includes pharmaceutical compositions and formulations which include one or more of the agents of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration; or oral administration; or via a spinal tap. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Clearly, the formulation needs to enable the agent to cross the blood brain barrier. Hence, the agent itself may need to be modified. Alternatively, the formulation may enable retrograde transport.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients.

Emulsions are typically heterogenous systems of one liquid dispersed in another in the form of droplets usually exceeding 0.1 ₗam in diameter. Emulsions may contain additional components in addition to the dispersed phases, and the active drug which may be present as a solution in either the aqueous phase, oily phase or itself as a separate phase. Microemulsions are included as an embodiment of the present invention. Emulsions and their uses are well known in the art and are further described in U.S. Pat. No. 6,287,860, which is incorporated herein in its entirety.

Formulations of the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

In one embodiment, the present invention employs various penetration enhancers to effect the efficient delivery of nucleic acids. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in vitro and in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years.

The present invention provides a neuroprotective formulation comprising galanin or a homology, derivative, analog or mimetic thereof and/or a galanin receptor agonist and one or both of LIF and/or CNTF or a homology, derivative, analog or mimetic thereof and one or more pharmaceutically acceptable carriers and/or diluents.

In another embodiment, the present invention is directed to a neuroprotective formulation comprising galanin and/or a galanin receptor agonist and one or both of LIF and/or CNTF and one or more pharmaceutically acceptable carriers and/or diluents.

The present invention further enables diagnostic assays for neurodegenerative disorders. One such assay is to measure for levels of galanin or galanin receptors alone or in combination with assays, for LIF and CNTF. Alternatively, magnetic resonance imaging (MRI) may be employed, such as of the optic nerve.

Hence, the present invention contemplates MRI as a surrogate marker of optic neuritis or other neurodegenerative conditions. The optic nerve represents a discrete anatomical structure comprised of a single white matter tract, oriented in one plane and which locks neuronal cell bodies.

MRI is particularly useful in animal model studies.

Hence, the present invention extends to an animal model of a neurodegenerative condition comprising subjecting a non-human animal induced to exhibit experimental immune encephalomyelitis (EAE) to magnetic resonance imaging (MRI) before and after administration of a potential neuroprotective agent or formulation comprising neuroprotection agents.

The optic nerve is also an ideal focus for study from a clinical research and development perspective. First, it has been estimated that some 40% of relapses of MS involve the development of secrete inflammatory lesions within the optic nerve, resulting in optic neuritis. Although visual acuity usually recovers, persistent axonal injury is the rule, as revealed by residual visual field defects, disturbances of contrast recognition and clinical visualization of optic nerve head pallor and atrophy. Secondly, the histopathology of optic neuritis is similar to that which occurs in MS elsewhere in the neuraxis, indicating it is likely that what is applicable in optic neuritis from a therapeutic perspective can be extrapolated to cerebral and spinal cord components of the disease. Thirdly, optic nerve function can be more reliably assessed by quantitative clinical and electrophysiological measures than is possible in other white matter tracts, providing the opportunity to develop composite approaches to the assessment of efficacy of therapeutic agents in this context.

It has been recognized for some years that optic neuritis is a common site of tissue damage in EAE. One of the most commonly studied mouse strains used for induction of EAE is the C57B16 mouse with disease induced by an encephalitogenic epitope (MOG35-55) of the myelin oligodendrocytic glycoprotein, popularized because of its reasonable approximation to human MS.

Accordingly, the present invention contemplates optic neuritis, such as it occurs in EAE as a model to test the efficacy of neuroprotective/regenerative agents, including LIF, galanin, and CNTF. The efficacy of such agents is tested primarily by histopathological analysis and MRI as a surrogate measure of neuropathology. The capacity to test efficacy in a context that is translatable both conceptually and methodologically via use of the same surrogate markers in phase II trials of human MS.

The present invention is further described by the following non-limiting Examples.

### Example 1

### Determination of the Efficacy of LIF in Ameliorating the Oligodendroglial and Axonal Pathology of Optic Neuritis

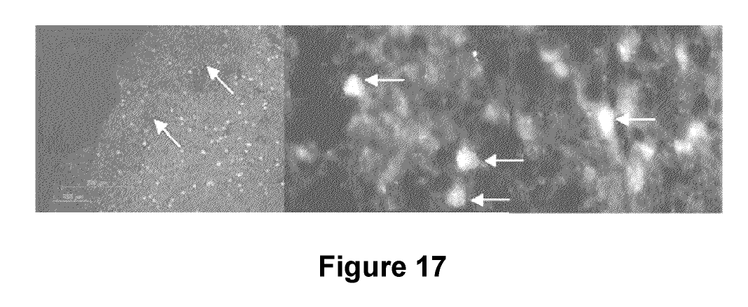

Experimental autoimmune encephalomyelitis is induced in inbred mice on the C57B16 background of 8-10 weeks of age by immunization with MOG35-55 peptide according to standard protocols. See, for example, Slavin et al, Autoimmunity 28:109-120, 1998 and Butzkeuven et al, supra 2002. For these experiments, mice that carry the PLP-dsRed transgene (Hirrlinger et al, Mol Cell Neurosci 30:291-303, 2005) are used. This mutant mouse is healthy and fertile and has no developmental phenotype but carries a red fluorescent marker that specifically identifies and allows easy quantification of oligodendrocytes. This transgenic animal develops EAE (see FIG. 17). Cohorts of 20 mice were analyzed, 10 of which received vehicle only as controls and 10 of which received LIF subcutaneously in a dose of 25 μg/kg/day in 0.1% w/v Mouse Serum Albumin (MSA), representing a regimen that have previously found to be efficacious in ameliorating the clinical severity of EAE (Butzkueven et al, supra 2002).

Two treatment schedules are carried out. In schedule one, the mice receive parenteral LIF or vehicle only injections from the day of disease induction and, in the second schedule, animals commence treatment after onset of paralytic disease, around day 12 (the exact timing in any given cohort depending on the tempo of onset of clinically overt spinal cord disease). The animals receiving treatment schedule one are sacrificed on day 20, and animals receiving delayed treatment are sacrificed after receiving seven daily doses of LIF/vehicle.

At the endpoint of the experiment, all animals are anaesthetized and have optic nerve MRI performed to determine the parallel ADC value in the prechiasmal portion of at least one or, whenever possible, both optic nerves. Immediately after the MRI scan, all animals are transcardially perfused with 4% v/v paraformaldehyde (PFA) in phosphate-buffered saline (for frozen sections) or 2.5% w/v glutaraldehyde/4% v/v PFA in sodium cacodylate buffer (for plastic embedding) and the optic nerves dissected out. Histological evaluation in separate cohorts includes:
1) 10 micrometer fresh-frozen longitudinal sections to assess the extent of inflammation (CD3 staining, DAPI-visualized infiltrate numbers and area) and oligodendrocyte number via cell lineage specific expression of the PLPdsRED transgene and fluorescence miroscopy.
2) 500 nanometer cross-sections from the prechiasmal area of plastic-embedded optic nerves to assess cross-sectional nerve size, total axon number, cross-sectional axolemmal area and axolemmal size distributions (for detection of axonal atrophy).

Detailed Histological Analysis

Oligodendrocyte numbers are assessed within optic nerves isolated from the LIF-treated and control animals by counting DS red positive cells in the longitudinal sections. It has been established that DS red is detectable in demyelinated tissue and approximately 150 oligodendrocytes can be delineated per longitudinal optic nerve section in the normal adult. Previous results utilizing MBP-lacZ mice have indicated that there is approximately a 30% reduction in the density of oligodendrocytes within the spinal cords of animals with established EAE and that the administration of LIF fully rescued this phenotype. Analysis of optic nerves from 10 animals is sufficient to detect a 30% difference in oligodendrocyte number in this context. Preliminary results studying the optic nerves of PLPdsRed transgenic mice indicate that there is at least a 50% reduction in the numbers of dsRed positive cells in EAE, indicating a useful baseline from which to assess the therapeutic efficacy of LIF in this context. For completeness, the number of infiltrating T-cells is assessed. Staining with polyclonal anti-CD3 antibodies (DAKO, Denmark) is used to quantify numbers of infiltrating T-cells. As an additional measure of immune cell infiltration, DAPI-positive infiltrates in each nerve (Butzkueven et al, supra 2006) is determined.

An analysis of ultra-structural changes in transverse sections of optic nerves isolated from 10 LIF treated and 10 control animals is also be undertaken on tissues harvested from one cohort of mice given either vehicle or LIF for 20 days. Three cross-section 0.5-micron resin-embedded methylene blue-stained serial optic nerve sections (each separated by 50 micron) is used to quantify nerve size, axon number, cross-sectional axolemmal area and axolemmal atrophy (assessed by axolemmal area frequency distributions). Standard electron microscopy is used for qualitative analysis of morphological abnormalities. Focus of the analysis of axonal pathology on the pre-chiasmal region of the nerves is undertaken, as this area shows maximal inflammatory infiltration, and has also been shown to consistently exhibit maximally reduced lambda parallel ADC-values in the preliminary analyses.

Statistical Analysis

All parametric data is analyzed using one way ANOVA with the Tukey-Kramer multiple comparison post-test as required. Correlations is assessed using Pearson correlation coefficients. EAE grades are compared using the Mann-Whitney U-test for nonparametric comparisons. All statistical analyses are performed using InState V3.0b for Macintosh (San Diego, Calif.).

Assessment of the Role of Endogenous Galanin in Ameliorating the Oligodendroglial and Axonal Pathology of Optic Neuritis

Initially, the optic nerves is assessed by prechiasmal parallel DTI (MRI), as well as optic nerve size, the number of PLPdsRED positive cells, axon number and axolemmal size distribution in n=8 (4 male, 4 female) healthy galanin−/−PLPdsRED+ve and galanin+/+PLPdsRED+ve animals to ensure no subtle, basal phenotype exists. Analysis of the severity of oligodendroglial and axonal pathology in EAE-associated optic neuritis in 10 animals from each genotype using an identical experimental plan as outlined above. The analysis is conducted on day 20 post-induction. Initially, 10 animals are assessed with optic neurities from each genotype by MRI (parallel ADC) and assess axonal pathology and nerve size utilizing plastic embedded, methylene-blue stained 0.5 micron sections as detailed above. A second replicate cohort is also assessed by MRI but then processed for longitudinal frozen optic nerve sections to characterize the extent of inflammatory infiltration and to perform dsRED cell counts.

Galanin knockout mice are proposed to exhibit either increased axonal injury or reduced oligodendrocyte numbers in optic neuritis. Hence, the GalR1 and GalR2 knockout animals are examined in an identical paradigm to further examine the likely signaling pathway involved. Both knockout animals are available on the C57B6 background and are interbred with PLPdsRED animals if required.

Determination of the Reliability, Validity and Responsiveness of MRI-Based protocols to Detect Axonal Injury in the EAE Optic Nerve

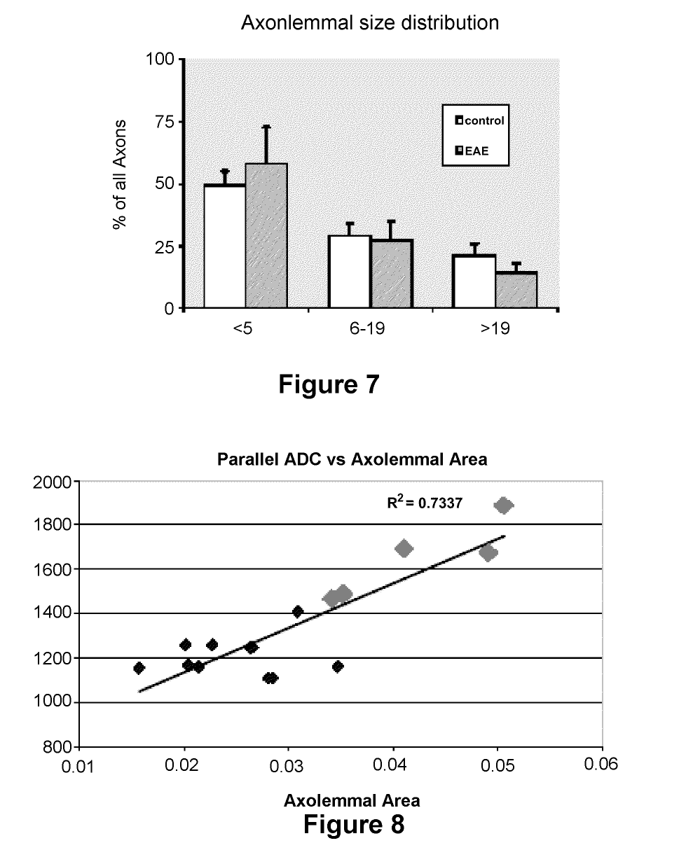

It has been established that parallel ADC in the prechiasmal portion of the optic nerve is consistently reduced in animals with relatively severe EAE, and that this correlates best with the reduction in total axolemmal cross-sectional area (FIG. 7). Although the primary endpoints of the above studies of the potential therapeutic effect of LIF and galanin are histological, it is proposed that axonal protection, afforded by LIF or galanin, is also detectable by increased diffusion of water along the long axis of nerve is made. Therefore, these experiments provide an opportunity to establish MRI as a surrogate marker for axonal injury by assessing milder forms of axonal pathology and correlate these with parallel ADC values. In addition, to acquisition of MRI data for the experimental cohorts described above, study of n=5 wild-type C57B6 mice of each gender at day 15 and 18 post EAE-induction (MOG 35-55) is also carried out. Mice are perfused with 2.5% w/v glutaraldehyde/4% v/v PFA in sodium caodylate buffer for plastic embedding of the optic nerves and assessment of axonal pathology on methylene-blue stained prechiasmal cross-sections as described above (FIGS. 6 and 7). In this way, the temporal profile of axonal damage in this model is established and the correlation between MRI measures and the relevant histopathology determined.

Detailed MRI Protocol

The mice are anaesthetized with 2-2.5% v/v isoflurane, placed in an MRI-compatible head holder with a nose cone placed over the snout and anaesthesia maintained with 0.5-2.0% v/v isoflurane. The core temperature and respiratory rate of the mice is monitored during the scanning process. Scanning is undertaken using a Bruker 4.7T MRI system. A mid sagittal slice of 1.0 mm thickness is acquired using a fast spin echo (FSE) sequence as a localizer. The bilateral junctions of the retina and optic nerves and optic chiasm are used to determine the orientation of the oblique slice containing the optic nerve from the retina bilaterally to the optic chiasm. A spin echo imaging sequence (ParaVision 3.01, Bruker) is used to acquire the diffusion-weighted images with only two different diffusion gradient directions, of which one is parallel and the other perpendicular to the selected optic nerve. Images are acquired with the following parameters: TR of 1 s, TE of 30 ms, Δ of 10 ms, 8 NEX, slice thickness 0.5 mm, field of view 2×2 cm², data matrix 256×128. B-values of 0 s/mm² (non-diffusion weighted image) are used, 700 s/mm² for the two diffusion sensitizing gradient directions. Two T2 weighted images using fast spin echo sequence (RARE, ParaVision, Bruker) is also used to measure the T2 of the optic nerve using TE 22 ms, 57.2 ms respectively, and the following parameters are: TR of 2 s, RARE factor of 2 for each image, 8 NEX, slice thickness of 0.5 mm, field of view of 2×2 cm², data matrix of 256×256.

Immediately after MRI scanning, the mice are killed by CO₂ inhalation and processed as described above. Regions of interest (ROIs) for the ADC analyses are manually traced on the T2 weighted image of the chiasm section of the optic nerve. This avoids any partial volume effect and signal contamination effect arising from the surrounding aft in the orbital section immediately behind the retina. The ROIs are then projected onto the T2-map and ADC images and used for quantification.

Correlations within the diseased cohort for degree of axonal degeneration, oligodendrocyte loss and degree of inflammation and ROI values delineated for parallel ADC value are assessed utilizing the Pearson product correlation coefficient. This work allows the collection of data from a sufficient number of untreated EAE animals (n=40) to also perform multivariate regression with forward selection of the most significant variables in order to compare the relative contribution of various histological measures with the prechiasmal optic nerve parallel ADC values.

Assessment of the Severity of Cuprizone-Induced Demyelination and Efficacy of Subsequent Remyelination in Galanin Knockout Mice and Galanin R2 Knockout Mice in Comparison to C57B6 Wild-Type Mice

In order to determine the significance of LIF-mediated galanin up-regulation during cuprizone-mediated demyelination in sublethally injured oligodendrocytes, cuprizone experiments are performed in galanin knockout mice and galanin-receptor-2 knockout mice. Each experimental group contains six animals/genotype and all experiments are conducted in duplicate.

Cuprizone-mediated demyelination is induced by feeding the mice a diet of powdered fee (Barastoc) containing 0.2% w/w cuprizone (Sigma) for either 3 or 4 weeks. Myelin density is assessed and oligodendrocyte numbers at 3 and 4 week time-points using at least three separate paraffin sections selected for each animal at or adjacent to Bregma −0.75 mm. Myelin density is assessed using Luxol Fast Blue (LFB) staining. Sections are post-fixed in 10% v/v buffered formalin solution for 10 minutes, placed in LFB solution (1 g/titre of Luxol Fast Blue dye, Merck, Darmstadt, Germany, in 95% v/v ethanol containing 0.05% w/v acetic acid) for 2 hours at 60° C., washed for one minute in running tap water, differentiated by alternative washes in saturated Lithium Carbonate (0.05% w/v) and 70% v/v ethanol (5 times in each solution), again washed in tap water, dehydrated and cleared in graded ethanol and histolene, and finally coverslipped using DPX.

For densitometric analyses to quantitated myelination, sections are photographed at ×10 objective and the images imported into Adobe Photoshop. Within Photoshop the blue channel of each image are selected and exported as a greyscale image into NIH Image Analysis 1.63 in order to take a density reading of the LFB staining within the central 870 μm of the corpus callosum. Density measurements for each mouse are normalized to values between 0 and 100 using the lowest reading (most severely demyelinated) as 0 and the highest reading (noncuprizone treated mouse) as 100, using the formulae Transformed value=((density reading-lowest density read)/highest density reading-lowest density reading)×100. All analysis are performed blind to the genotype and treatment of the animal and analyzed using one-way ANOVA (Emery et al, supra 2006).

GST-Pi (ABCAM) staining are utilized to analyze oligodendrocyte numbers from paraffin sections adjacent to those used for myelin densitometry. Sections are counter-stained with DAPI nuclear stain and photographed at ×200 magnification at the midline of the corpus callosum (corresponding to the medial 425 μm of each section). The number of GST-Pi immunopositive cells with DAPI positive nuclei within the corpus callosum is counted for each image and the area of the corpus callosum determined using the NIH Image 1.63 program. All counts and analyses are performed blind to the genotype and treatment of the animal.

For assessment of remyelination, six animals of each of the above genotypes are fed cuprizone (0.2% w/v) for 4 weeks, followed by 4 weeks of normal diet. After sacrifice, the brains are processed for paraffin embedding and undergo assessment of myelin density and oligodendrocyte density, as described above.

Examination of Whether the Known Beneficial Effect of Exogenous LIF in Reducing Demyelination in the Cuprizone Model is Dependent on Galanin

It is proposed that galanin knockout mice and galanin receptor-2 knockout mice exhibit an enhanced demyelinating phonotype in the context of cuprizone administration. Additional cuprizone experiments are, therefore, conducted. Animals of each of these genotypes and wild-type controls are treated with daily LIF (50 μg/kg/day subcutaneously) or placebo for 4 weeks. It is proposed that the protective effect of LIF is mediated via stimulation of galanin secretion and/or galanin receptor-2 activation, recombinant LIF should, therefore, lose its protective effect in mice of these genotypes undergoing cuprizone demyelination. This experiment is conducted in duplicate with n=20 mice per genotype (wild-type, galanin ko, GalR2 ko) 10 of which receive LIF treatment and 10 of which will receive placebo treatment. Outcome measures include LFB density in the caudal corpus callosum and GST-pi positive oligodendrocyte density in the same area.

### Example 2

### MRI Correlations with Optic Nerve Pathology in EAE

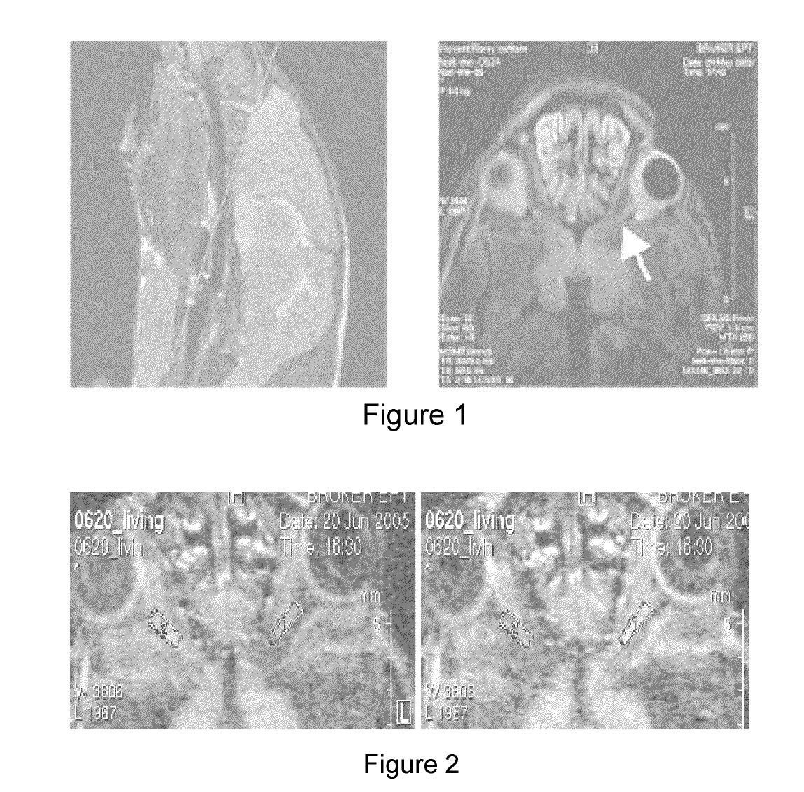

Experimental autoimmune encephalomyelitis (EAE) induced with MOG35-55 in C57B16 mice causes inflammatory demyelination in the spinal cord and optic nerves. Utilizing a high field strength magnet high quality images are obtained of optic nerve for T2 mapping and Diffusion Weighted Imaging (FIGS. 1 and 2) in anaesthetized mice. The methodology is explained in detail in Example 1.

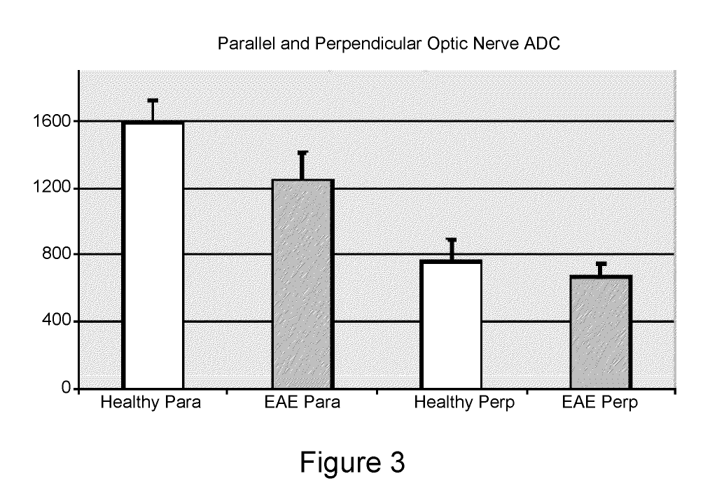

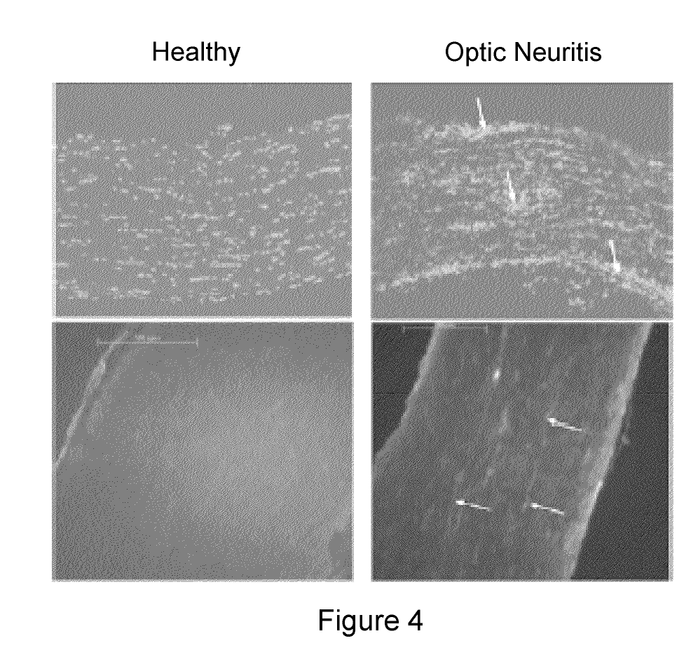

The parallel and perpendicular Apparent Diffusion Coefficient (ADC) is measured in 20 mice suffering EAE and in 10 healthy age matched controls. Mice with significant hindlimb involvement (Grade 2.5 or more; Butzkueven et al, supra 2002) were scanned between day 20 and 25 after EAE induction. The nerve was divided into two segments of approximately equal length, a retro-orbital and a pre-chiasmal portion, in order to allow parallel and perpendicular diffusion direction fits, as most nerves are slightly curved. The pre-chiasmal portion showed a 20% reduction in parallel ADC in the EAE mice (FIG. 3). On the other hand, no significant change in perpendicular ADC between normal and EAE mice could be detected.

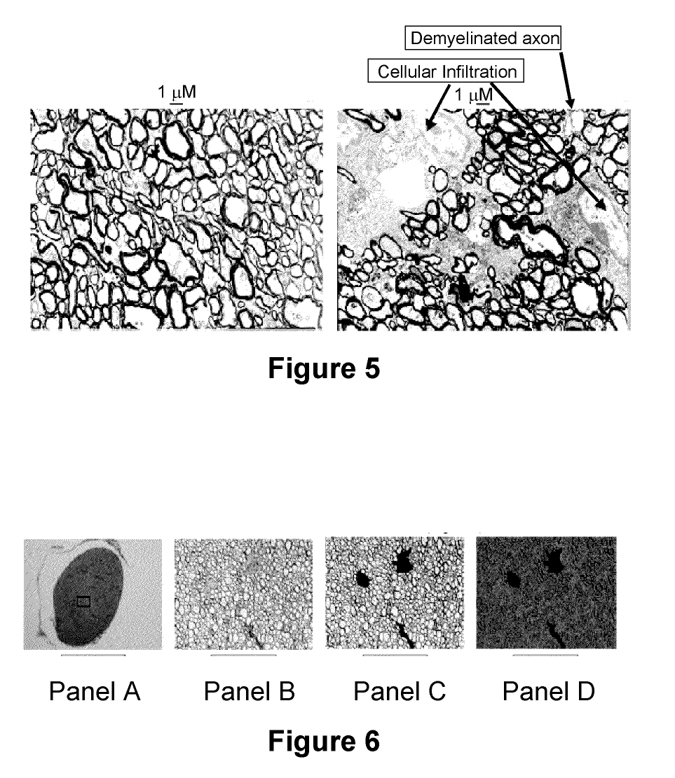

The extent of demyelination in the EAE-affected optic nerves was quantitated by counting the percentage of demyelinated axons from plastic-embedded thin cross-sections (pre-chiasmal region) processed for electron microscopy (8000 times magnification). Interestingly, although marked cellular infiltration/proliferation was readily discernible, very few demyelinated axons were observed in 5 EAE optic nerves each examined in four different areas (FIG. 5). In this context, the identification and nomination of a structure as an axon required the presence of either axolemmal granules or mitochondria. A rare example of a demyelinated axon is seen in FIG. 5, right panel.

Axonal pathology was quantitated in this model by examining plastic-embedded 0.5 micron optic nerve cross-sections, taken from the pre-chiasmal region. Sections were stained with the myelin stain methylene blue, and the central portion of each nerve was photographed at 100 times magnification. Images were imported into Image J 1.86 (NIH freeware) and an automatic threshold function was used as illustrated to delineate the axolemma (FIG. 6).

Using five healthy nerves and 10 nerves from animals suffering severe EAE (paraplegia score 2.5 or greater), nerve area measurements from sections at 20 times magnification were obtained (FIG. 6, Panel A). In EAE, the total nerve area was reduced by an average of 20%, from an average of 0.08 mm² to 0.064 mm² (p<0.02). Total estimated optic nerve axonal number was reduced by 24%, from 12480 axons/nerve in healthy mice to 9431 axons/nerve in EAE (p<0.003). Interestingly, the axonal measure most severely affected in optic neuritis was the total axolemmal cross-sectional area, which was reduced by 41% (from 0.035 mm² to 0.020 mm², p<0.001). In concordance with this result, examination of axolemmal size frequency distributions revealed axonal atrophy (FIG. 7).

Univariate correlations were examined between histological/clinical measures of axonal injury and parallel ADC in the prechiasmal optic nerve region. Total axon number (r=0.59, p=0.02) and EAE Grade (r=0.7, p=0.01) correlated moderately well with parallel ADC. The tightest correlation was observed between total cross-sectional axolemmal area and parallel ADC (FIG. 8).

### Example 3

### Functional Significance of LIF-Dependent Oligodendroglial Galanin Up-Regulation in Cuprizone-Induced Demyelination

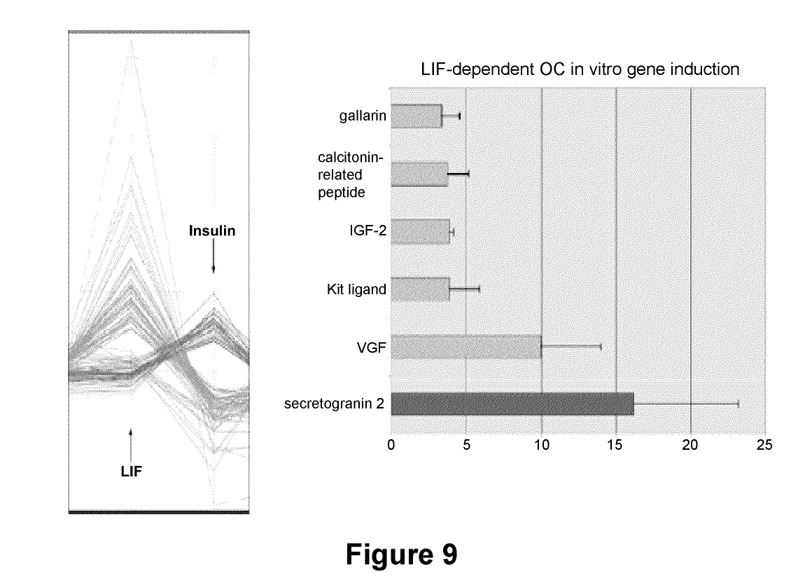

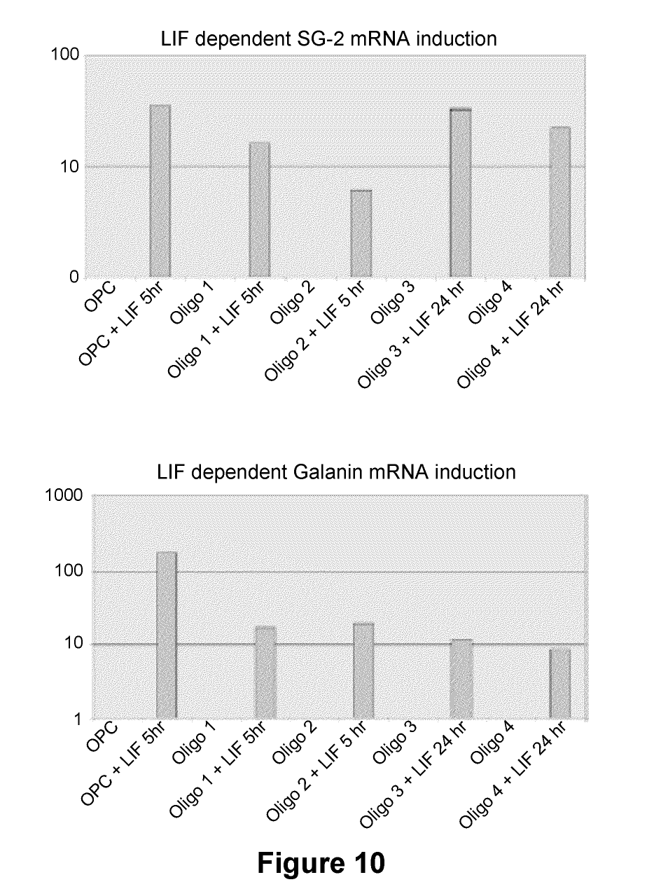

In order to assess mRNA induction promoted by LIF signaling in oligodendrocytes, an Affymetrix array analysis was conducted of cultured primary oligodendrocytes. A2B5 positive, O1 negative oligodendrocyte progenitor cells were isolated by sequential immunopanning from P6 rat optic nerves. After expansion in culture (2 passages, 11 days) in Sato medium in the presence of insulin, platelet-derived growth factor (PDGF) and neurotrophin-3 (NT3) (no CNTF or LIF), the cells were matured by 48 hours of PDGF withdrawal. The NT3 was also withdrawn for this time-period but insulin was maintained. In order to determine gene expression profiles induced by various cytokines, the matured cells were then exposed to one of four conditions for 8 hours: Sato alone, Sato plus insulin, Sato plus LIF, Sato plus NT3. Subsequently, the cells were harvested, mRNA isolated and processed for Affymetrix gene chip hybridization (Rat 230 2.0). The experiments were conducted as independent triplicates. Potentially regulated genes were selected for further analysis if they exhibited at least 1.3 fold regulation in any one comparison. The 965 genes identified were subjected to cluster analysis. Distinct clusters of genes were regulated by insulin and LIF (FIG. 9) but not NT-3. The identity of several LIF-regulated genes suggested the induction of neuropeptide/cytokine secretion in oligodendrocytes.

For each of these six regulated genes, independent confirmation was conducted by real-time PCR. Either dividing oligodendrocyte progenitors or mature oligodendrocytes (as above) were exposed to LIF for either 5 or 24 hours, and measured gene induction against baseline (independent baseline cultures for each comparison). All six genes were confirmed to be regulated at the RNA level. FIG. 10 illustrates typical results for secretogranin 2 and galanin.

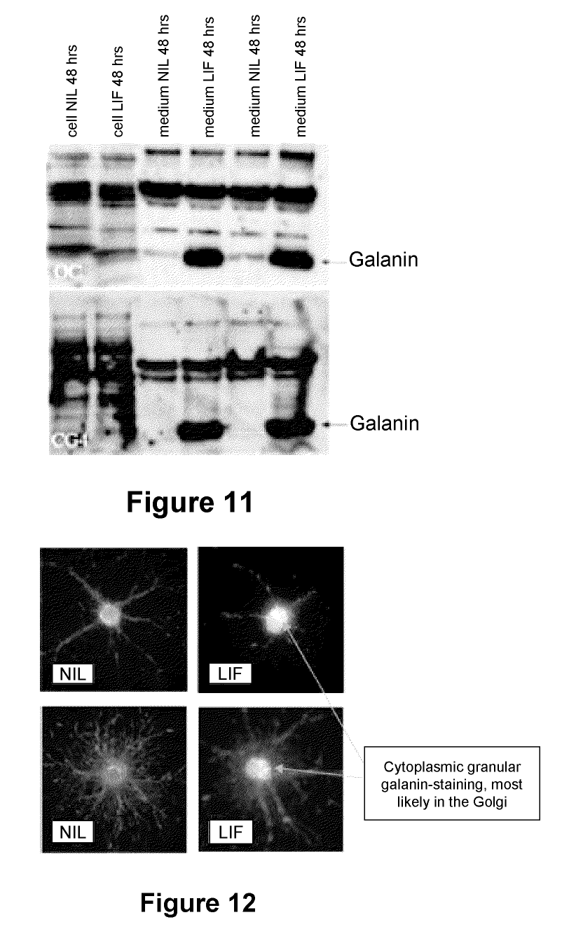

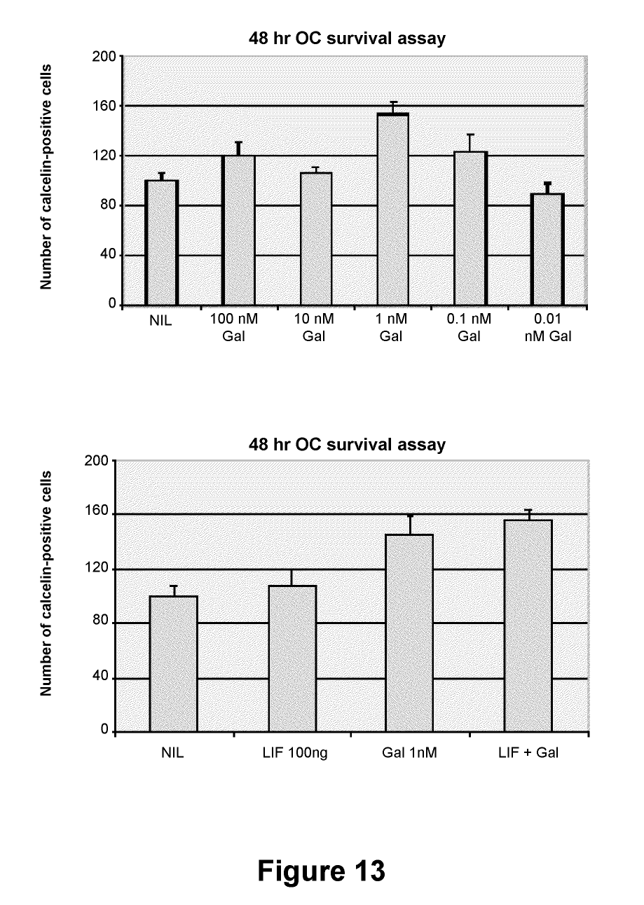

Translation of regulated cytokine and neuropeptide mRNA species into peptides/proteins was confirmed. Mature oligodendrocytes (as above) were plated in 10 cm dishes in SATO plus insulin and added LIF (100 ng/ml) to half the dishes for 48 hours. Induction of galanin production was observed in concentrated media (FIG. 11). LIF-dependent galanin production in concentrated media from the OC cell line CG4 (FIG. 11) was confirmed. Consistent LIF-dependent regulation of the other cytokines in the retina (CRP, VGF, IGF2, kit ligand) nor of secretogranin-2 at the protein level was not observed. The induction of galanin production in response to LIF was not observed in cultured primary rat astrocytes or an n-mye immortalized astrocytic cell line, strongly suggesting lineage-specificity for this response. All Western blots were performed in triplicate from independent experiments. The LIF-dependent induction of a secretory phenotype in OC was confirmed by galanin immunocytochemistry of OC cultured in insulin +/− LIF for 48 hours (FIG. 13).

In order to determine the potential for autocrine galanin signaling, the presence of the mRNA was evaluated for the three known galanin receptors in OC. The relative expression profiles were very discordant, with the mRNA for GalR2>GalR3>GalR1. Relative to GalR1, the mRNA level for GalR3 was 32 fold higher, and GalR2 was 2000 fold higher. Results for the CG4 oligodendroglial cell line were very similar. Therefore, the potentiality of autocrine signaling of galanin in OC via GalR2 or GalR3 exists.

Whether or not galanin itself is a survival factor for oligodendrocytes in vitro was determined. Primary rat OPC in culture were matured for 48 hours by PDGF withdrawal in the presence of insulin, passaged into poly-1-lysine coated 24 well-plates at a density of 200 cells/well in Sato medium alone or Sato medium supplemented with various concentrations of recombinant rat galanin (Peninsula Biosciences).

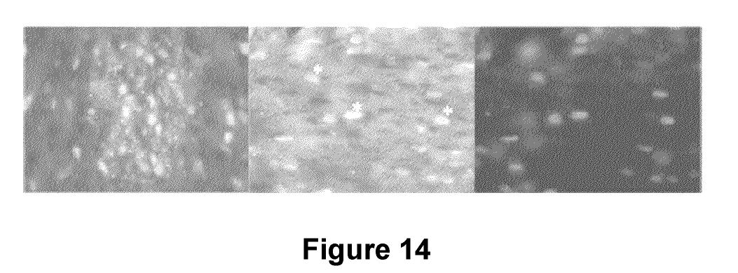

Twenty wells/condition were counted in each survival assay and results were verified in four independent experiments using separate batches of primary cells. All counts were performed by an investigator blinded to the experimental condition. A cell was defined as alive if it displayed rapid uptake of calcein (green, live cell stain) and no staining with ethidium homodimer (red, dead cell stain). Typical results are shown in FIG. 14, revealing a highly significant survival effect at a low concentration of galanin (1 nM), which is abolished at higher concentrations.

The concentration-dependent survival effect of galanin, with promotion of OC survival only at low doses could reflect differential activation of GalR2 and GalR3 in the cells and is subject to further investigation, independent of this application. Importantly, the galanin-mediated OC survival effect is not synergistic with LIF, which could signify that the mechanism of LIF-mediated OC survival is dependent on LIF-induced galanin secretion.

The expression of LIF and CNTF are known to be up-regulated in the corpus callosum during cuprizone-induced demyelination and LIF knockout mice suffer more severe demyelination and oligodendrocyte loss (Emergy et al, supra 2006). Whether or not galanin induction in the corpus callosum during cuprizone-mediated demyelination was a) detectable and b) LIF and/or CNTF dependent was determined. Transgenic mice expressing the fluorophores dsRED under the control of the PLP promoter (PLP-ds red) (Hirrlinger et al, supra 2005) were subjected to cuprizone for 3 weeks. The mice developed typical caudal corpus callosal oligodendrocyte loss and macrophage/microglial infiltration. Galanin staining of coronal frozen sections of caudal corpus callosum revealed induction of galanin staining in dsRED-positive oligodendrocytes (FIG. 14).

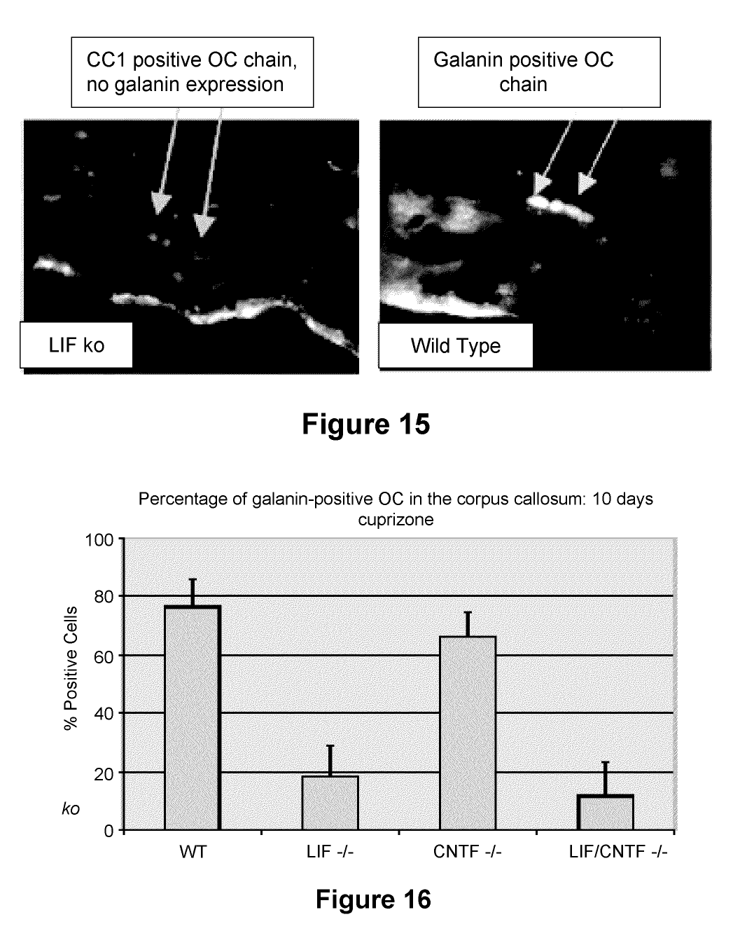

In order to determine whether galanin production by OC during cuprizone demyelination ws LIF-dependent, 6 wild-type C57B6, 6 LIF knockout, 6 CNTF knockout and 2 LIF/CNTF double ko mice were subjected to cuprizone for 18 days, an early time-point at which LIF and CNTF are already induced in the corpus callosum (Emery et al, supra 2006) but at which overt demyelination is minimal. The percentage of galanin+ve cells in the central corpus callosum marked with the oligodendrocyte-specific anti-CC1 antibody (FIG. 15) were counted. Galanin was already induced in CC1-positive cells after 18 days of cuprizone challenge and was clearly dependent on endogenous LIF but not CNTF production (FIG. 16).

PLPdsRED transgenic mice were used for optic neuritis experiments hence whether these mice have a similar EAE phenotype as wild-type C57B16 mice was verified. A cohort of n=5 female and n=5 male PLPdsRED transgenic mice developed EAE with onset on day 12, and mean disease severity of 2.75 (complete tail paralysis, almost complete hind-limb paralysis), range 1.5-4.0 by day 22. There was no gender difference. Utilizing dsRED positive cells to identify oligodendrocytes, it was demonstrated that the regulated expression of galanin in oligodendrocytes within inflammatory infiltrates and pen-infiltrate areas in which approximately 50% of PLPdsRED positive cells contained galanin-positive granules (FIG. 17).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Banner et al, Exp Neurol 147:1-9, 1997

Barres et al, Cell 70:31-46, 1992

Barres et al, Development 118:283-295, 1993

Butzkeuven et al, Nat Med 8:613-619, 2002

Butzkeuven et al, Glia 53:696-703, 2006

Canoll et al, Neuron 17:229-243, 1996

D'Souza et al, J Neurosci Res 43:289-298, 1996

Emery et al, Proc Natl Acad Sci USA 103(20):7859-7864, 2006

Ernst et al, J Exp Med 194:189-203, 2001

Fernandez et al, Neuron 28:81-90, 2000

Hirrlinger et al, Mol Cell Neurosci 30:291-303, 2005

Hodgson, Bio Technology 9:19-21, 1991

Johnson et al, Peptide Turn Mimetics in Biotechnology and Pharmacy, Pezzuto et al, Eds., Chapman and Hall, New York, 1993

Linker et al, Nat Med 8:620-624, 2002

Lucchinetti et al, Brain 122:2279-2295, 1999

Lucchinetti et al, Ann Neurol 47:707-717, 2000

Marriott et al, LIF receptor signaling modulates myelin loss and repair during cuprizone demyelination, Poster Abstract, The Australian Neuroscience Society 26^{th} Annual meeting, 2006

Mayer et al, Development 120:143-153, 1994

Slavin et al, Autoimmunity 28:109-120, 1998

Suzuki et al, Lab Invest 20:444-454, 1969

Trapp et al, N Engl J Med 338:278-285, 1998

Vartanian et al, Proc Natl Acad Sci USA 96:731-735, 1999

Vercellino et al, J Neropathol Exp Neruol. 64:1101-1107, 2005

Ware et al, Development 121:1283-1299, 1995

1. A method for the treatment or prophylaxis of a neurodegenerative disease or condition in a subject, said method comprising administering to said subject an effective amount of galanin or homolog, derivative, analog or mimetic thereof and/or an agent which up-regulates the level or activity of a galanin receptor for a time and under conditions sufficient to promote survival and/or maintenance of oligodendrocytes, inhibit demyelination and/or promote axonal and/or neuronal repair and function.

2. The method of claim 1 further comprising the administration of a neuroprotective agent.

3. The method of claim 2 wherein the neuroprotective agent is LIF or CNTF or both or a homolog, derivative, analog or mimetic thereof.

4. The method of claim 1 wherein the galanin receptor is galanin receptor 1, galanin receptor 2 or other member of the galanin receptor family.

5. The method of claim 1 comprising the administration of galanin.

6. The method of claim 1 wherein the subject is a human.

7. The method of claim 1 wherein the neurodegenerative condition is selected from the list consisting of MS, acute disseminated encephalomyelitis, optic neuropathy (including neuromyelitis optic with transient autonomic disturbances) Devic's neuromyelitis optica, tropical spastic paraparesis, non-compressive myelopathies, concentric sclerosis, diffuse sclerosis acute hemorrhagic leukoencephalopathy, metabolic leukodystrophy leucoarliusis, acute discriminated encephalomyelitis, progressive multifocal leukoencephalopathy, multisystem entrophy and repairing the demyelination associated with disease or trauma.

8. The method of claim 7 wherein the condition is MS.

9. A method for the treatment or prophylaxis of a neurodegenerative disease or condition in a subject, said method comprising administering to said subject an effective amount of a neuroprotective formulation comprising one or both of galanin or a homolog, derivative, analog or mimetic thereof and/or a galanin receptor agonist and one or both of LIF and CNTF or a homolog, derivative, analog or mimetic thereof for a time and under conditions sufficient to promote survival and/or maintenance of oligodendrocytes, inhibit demyelination and/or promote axonal and neuronal repair and function.

10. The method of claim 9 wherein the galanin receptor is galanin receptor 1, galanin receptor 2 or other member of the galanin receptor family.

11. The method of claim 9 which comprises the administration of galanin.

12. The method of claim 9 wherein the subject is a human.

13. The method of claim 9 wherein the neurodegenerative disease or condition is selected from the list consisting of MS, acute disseminated encephalomyelitis, optic neuropathy (including neuromyelitis optic with transient autonomic disturbances) Devic's neuromyelitis optica, tropical spastic paraparesis, non-compressive myelopathies, concentric sclerosis, diffuse sclerosis acute hemorrhagic leukoencephalopathy, metabolic leukodystrophy leucoarliusis, acute discriminated encephalomyelitis, progressive multifocal leukoencephalopathy, multisystem entrophy and repairing the demyelination associated with disease or trauma.

14. The method of claim 13 wherein the neurodegenerative condition is MS.

15. A method for treating MS in a subject, said method comprising administering to said subject an effective amount of galanin or a homolog, derivative, analog or mimetic thereof and/or a galanin receptor agonist for a time and under conditions sufficient to ameliorate the symptoms of MS and/or to promote survival and/or maintenance of oligodendrocytes, inhibit demyelination and/or promote axonal or neuronal repair or function.

16. The method of claim 15 further comprising the administration of a neuroprotective agent.

17. The method of claim 16 wherein the neuroprotective agent is LIF or CNTF or both or a homolog, derivative, analog or mimetic thereof.

18. The method of claim 15 wherein the galanin receptor is galanin receptor 1, is galanin receptor 2 or other member of the galanin receptor family.

19. The method of claim 15 comprising the administration of galanin.

20. The method of claim 15 wherein the subject is a human.

21. A method for the treatment or prophylaxis of a MS in a subject, said method comprising administering to said subject an effective amount of a neuroprotective formulation comprising one or both of galanin or a homolog, derivative, analog or mimetic thereof and/or a galanin receptor agonist and one or both of LIF and CNTF or a homolog, derivative, analog or mimetic thereof for a time and under conditions sufficient to promote survival and/or maintenance of oligodendrocytes, inhibit demyelination and/or promote axonal and neuronal repair and function.

22. The method of claim 21 wherein the galanin receptor is galanin receptor 2.

23. The method of claim 21 comprising the administration of galanin.

24. The method of claim 21 wherein the subject is a human.

25. A neuroprotective formulation comprising galanin or a homology, derivative, analog or mimetic thereof and/or a galanin receptor agonist and one or both of LIF and/or CNTF or a homology, derivative, analog or mimetic thereof and one or more pharmaceutically acceptable carriers and/or diluents.

26. The neuroprotective formulation of claim 25 comprising galanin and a galanin receptor agonist.

27. The neuroprotective formulation of claim 25 wherein the galanin receptor is galanin receptor 1, galanin receptor 2 or other member of the galanin receptor family.

28. The neuroprotective formulation of claim 25 comprising galanin and LIF or CNTF.

29. The neuroprotective formulation of claim 25 comprising a galanin receptor agonist and LIF or CNTF.

30. An animal model of a neurodegenerative condition comprising inducing an experimental immune encephalomyelitis (EAE) in a non-human animal and subjecting the animal to magnetic resonance imaging (MRI) before and after administration of a potential neuroprotective agent or formulation comprising neuroprotection agents.
